(19) United States
(12) Patent Application Publication
(10) Pub. No.: US 2014/0318551 A1
(43) Pub. Date: Oct. 30, 2014

(54) Craniotomy Drape and Method of Simultaneously Draping a Sterile Barrier Over a Patient and Navigation Tracker

(71) Applicant: Contour Fabricators, Inc., Fenton, MI (US)

(72) Inventor: Kevin J. Daly, Windermere, FL (US)

(73) Assignee: Contour Fabricators, Inc., Fenton, MI (US)

(21) Appl. No.: 14/264,345

(22) Filed: Apr. 29, 2014

Related U.S. Application Data
(60) Provisional application No. 61/816,960, filed on Apr. 29, 2013.

(51) Int. Cl. A61B 19/08

(52) U.S. Cl. CPC A61B 19/081; A61B 19/08
USPC 128/853

(57) ABSTRACT

A sterile craniotomy drape and method of simultaneously draping a sterile barrier over a patient and a navigation tracker prior to performing a craniotomy procedure are provided. The sterile craniotomy drape has a flexible, sterile wall with opposite edges extending lengthwise between opposite ends. The wall is sized to substantially cover a patient lying on a patient support surface of an operating table. The wall has a cranial region adapted to be attached to the patients head and at least one opening adjacent the cranial region. A flexible, tubular sterile sleeve has an open end attached to the wall about the at least one opening. The sleeve extends away from the wall to a free end for receipt of a navigation tracker therein.

### CROSS-REFERENCE TO RELATED APPLICATION

This application claims the benefit of U.S. Provisional Application Ser. No. 61/816,960, filed Apr. 29, 2013, which is incorporated herein by reference in its entirety.

### BACKGROUND OF THE INVENTION

1. Technical Field

This invention relates generally to sterile surgical drapes, and more particularly to sterile surgical drapes for craniotomy procedures.

2. Related Art

It is well known that maintaining a sterile environment within the region of a surgical site is critical in order to minimize the risk of infection to a patient. This generally requires maintaining sterility above the elevation of a patient support surface of an operating table. This is particularly important in brain surgery or any craniotomy procedure requiring forming a surgical opening into a patient's skull. A craniotomy procedure involves cutting an opening in the skull of the patient, and then performing the surgical procedure inside the skull of the patient, e.g. removing a tumor, removing a blood clot (hematoma), controlling a hemorrhage from a weak, leaking blood vessel (cerebral aneurysm), repairing arteriovenous malformations (abnormal connections of blood vessels), draining a brain abscess, relieving pressure inside the skull, performing a biopsy, or otherwise inspecting the brain.

Prior to initiating the surgical procedure, the patient is placed on a support surface of an operating table and the head of the patient is located and fixed within a head clamp to prevent the patient's head from moving during the surgery. The head clamp, in addition to fixing the patient's head from movement, is commonly operably attached to a neuronavigation receiver, also commonly referred to as “navigator tracker.” Neuronavigation is sometimes referred to as image guided surgery (IGS), computer assisted surgery (CAS) and stereotactic navigation. The navigator tracker is fixed to a stabilizer arm, which in turn, is typically attached to the head clamp for selective adjustment therewith. As such, with the patient's head fixed in the clamp, the navigator tracker can be precisely adjusted and calibrated and then fixed in the desired position, thereby acting as a fixed datum to allow the surgeon to know the precise location of his/her instruments within the patient's skull. As the surgeon moves an instrument within the brain, the position of the instrument, with the assistance of the fixed navigator tracker, is precisely calculated by a computer. The computer then superimposes the position of the instruments as they are manipulated in surgery onto images of the brain anatomy displayed on a monitor, which in turn, allows the surgeon to see the precise, real-time positioning of the instruments in three dimensions. This, in essence, is a real-time global positioning system (GPS) for the surgeon.

In order to perform neuronavigation, the navigator tracker must remain in its intended position “as calibrated and fixed.” Maintaining the navigator tracker in its calibrated and fixed position can be inadvertently compromised after initially fixing the navigator tracker in its desired position. Inadvertent movement of the navigator tracker generally results from the current and generally complex process used to drape the stabilizer arm of the navigator tracker as well as the patient. Should the navigator tracker become inadvertently moved from its originally fixed location, the imagery obtain via assistance from the navigator tracker will be inaccurate, thereby complicating the surgical procedure. The current process followed in performing a craniotomy includes: fixing the patient's head in the clamp; draping the patient with a sterile drape and cutting a hole in the drape to fit over the stabilizer arm; attaching a non-sterile navigator stabilizer arm and navigator tracker to the head clamp; positioning, calibrating and fixing the navigator tracker in the desired location; removing the navigator tracker from the non-sterile stabilizer arm; draping the non-sterile stabilizer arm with a sterile sock-shaped drape; draping the patient with a sterile patient drape; cutting a hole in the patient drape and pressing the patient drape over the draped stabilizer arm; wrapping a sterile cloth around the draped stabilizer arm at the opening cut in the patient drape; and attaching a sterile navigator tracker to the draped stabilizer arm, wherein the sterile navigator tracker is intended to remain in precisely the same position as the navigator tracker as originally calibrated and fixed. During this tedious draping process, there are numerous opportunities to inadvertently introduce bacteria to the desired sterile arena, such as through the hole cut in the patient drape, and to move the navigator tracker from its originally calibrated and fixed position, particularly when positioning the patient drape over the stabilizer arm and wrapping a sterile cloth about the opening, as well as while reattaching the sterile navigator tracker to the stabilizer arm, for example.

### SUMMARY OF THE INVENTION

In accordance with one aspect of the invention, a sterile craniotomy drape is provided. The sterile drape includes a flexible, sterile wall with opposite edges extending lengthwise between opposite ends, wherein the wall is sized to substantially cover a patient lying on a patient support surface of an operating table. The wall has a cranial region adapted to be attached to the patients head and at least one opening adjacent the cranial region. A flexible, tubular sterile sleeve has an open end attached to the wall about the at least one opening. The sleeve extends away from the wall to a free end.

In accordance with another aspect of the invention, the free end of the sleeve is closed.

In accordance with another aspect of the invention, the free end of the sleeve is optically transparent.

In accordance with another aspect of the invention, the wall has a pair of the at least one openings, each of the openings being on opposite sides of the cranial portion, wherein a separate sleeve is attached about each opening and extends outwardly from the wall.

In accordance with another aspect of the invention, the sleeve is folded telescopically and unfolds telescopically.

In accordance with another aspect of the invention, the sleeve has at least one fastener adapted to maintain material of the sleeve in a circumferentially gathered configuration.

In accordance with another aspect of the invention, a method of simultaneously draping a sterile barrier over a patient and a navigation tracker for a craniotomy procedure is provided. The method includes providing a sterile patient drape having a sterile tubular sleeve extending outwardly therefrom. Then, registering the tubular sleeve with the navigation tracker and covering the patient with the patient drape. Further yet, unfolding the tubular sleeve over the navigator tracker.

In accordance with another aspect of the invention, the method can further include gathering material of the tubular sleeve about a stabilizer arm supporting the navigation tracker and releasably fixing the material in its gathered configuration with a fastener.

In accordance with another aspect of the invention, the method can further include providing the fastener used to gather the sleeve material as an attached member extending from the sleeve.

In accordance with another aspect of the invention, the method can further include attaching an open end of the tubular sleeve in sealed relation about an opening in the patient drape.

### BRIEF DESCRIPTION OF THE DRAWINGS

These and other aspects, features and advantages of the present invention will become more readily appreciated when considered in connection with the following detailed description of presently preferred embodiments and best mode, appended claims and accompanying drawings, in which:

FIG. 1 is a plan view of a sterile patient drape constructed in accordance with one aspect of the invention;

FIG. 1A is a plan view of a sterile patient drape constructed in accordance with another aspect of the invention;

FIG. 2 is an enlarged fragmentary view of the sterile patient drapes of FIGS. 1 and 1A;

FIG. 3 is a perspective view of a patient on the operating table prior to disposing the patient drape over the patient; and

FIG. 4 is a view similar to FIG. 3 with the patient drape of FIG. 1 disposed over the patient.

### DETAILED DESCRIPTION OF PRESENTLY PREFERRED EMBODIMENTS

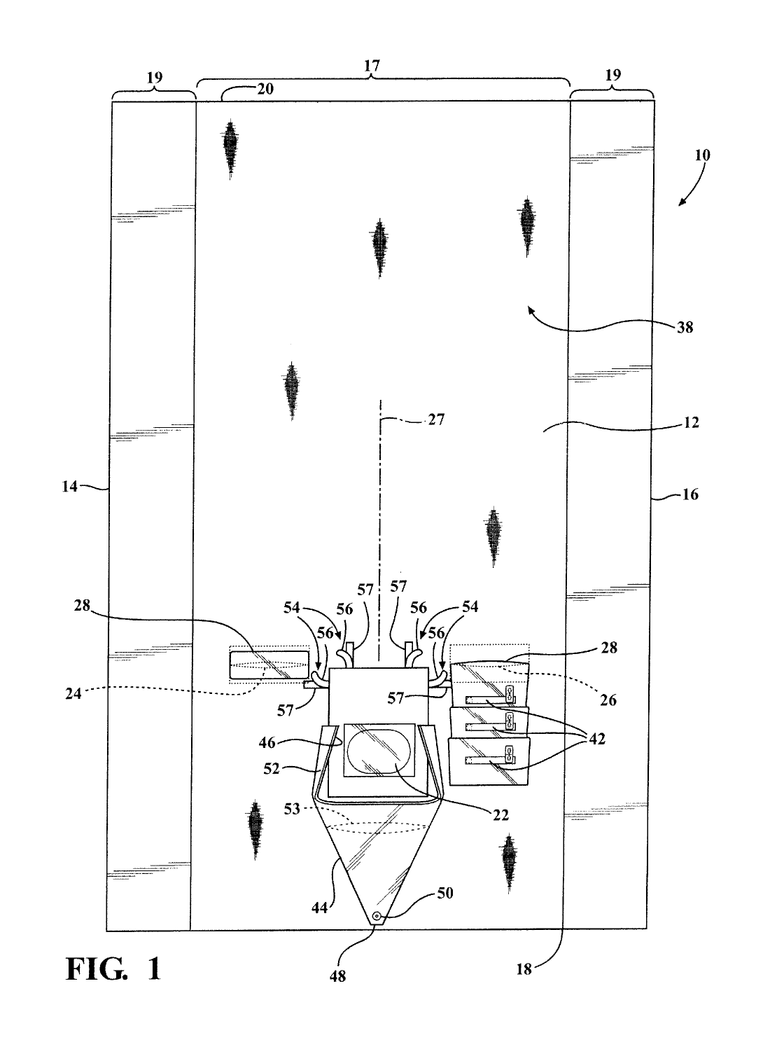

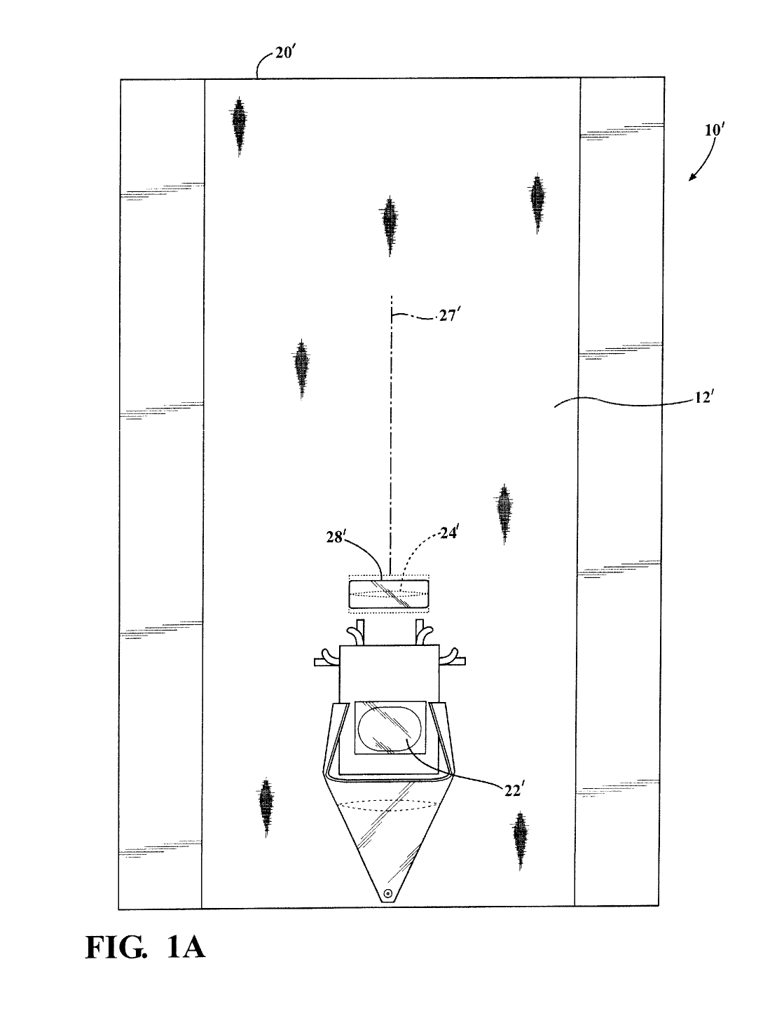

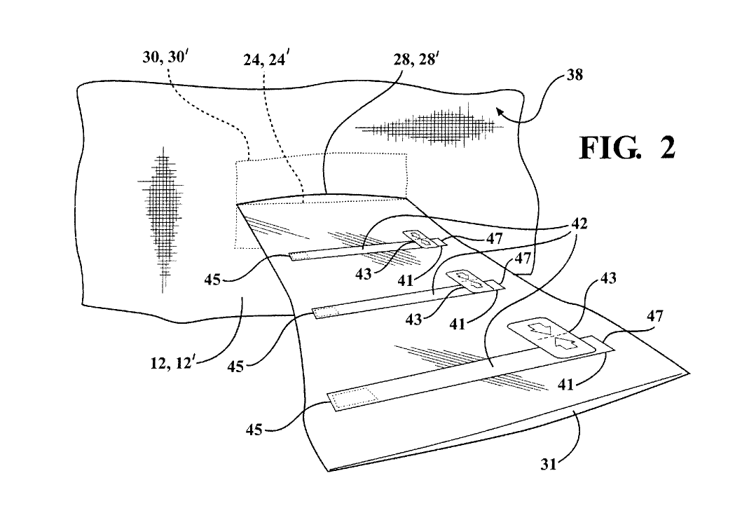

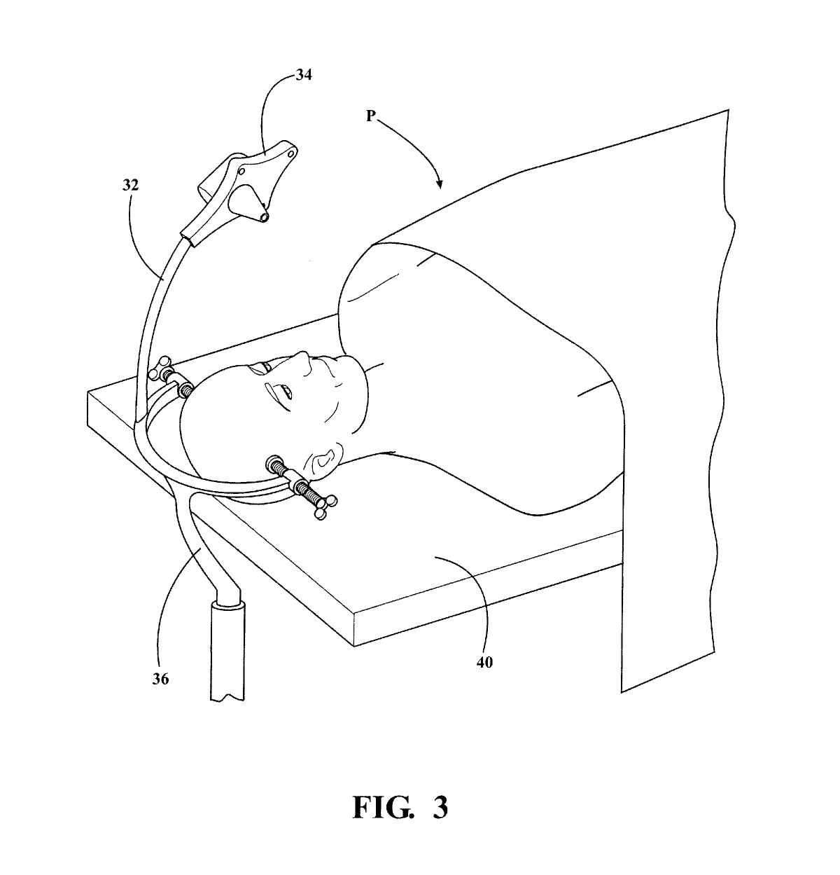

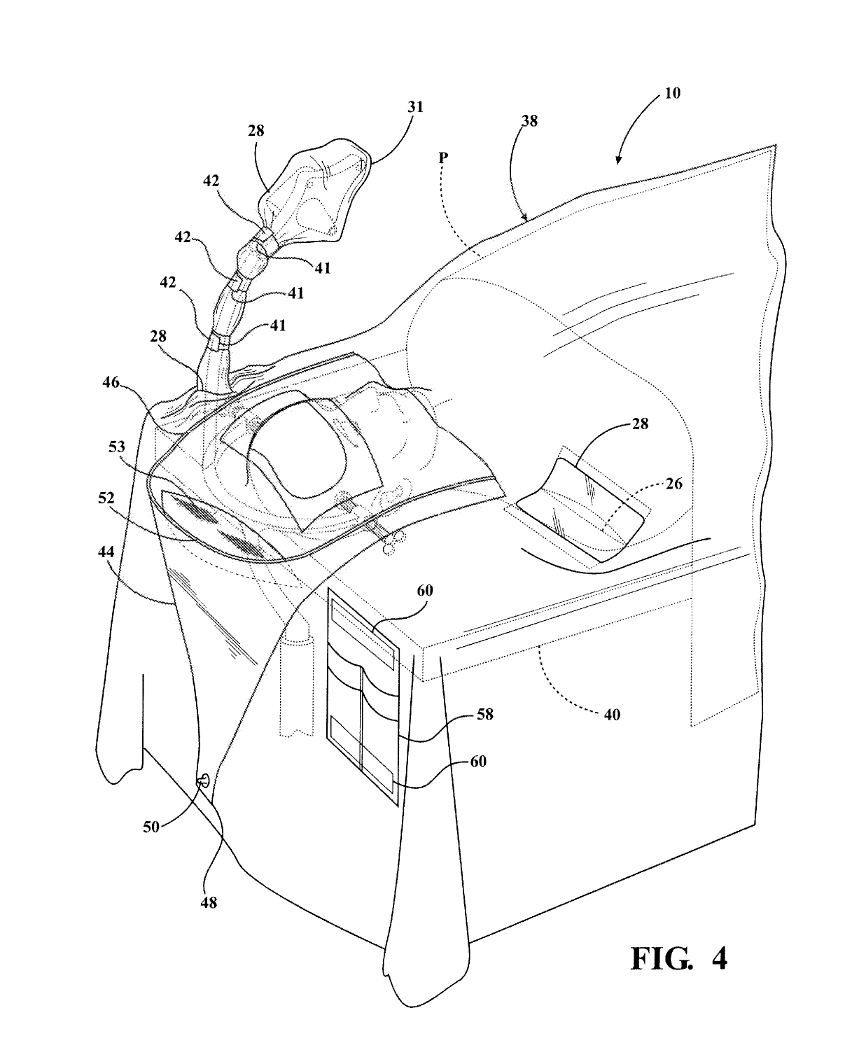

Referring in more detail to the drawings, FIG. 1 illustrates a sterile craniotomy drape, also referred to as patient drape or drape 10, constructed in accordance with one presently preferred embodiment of the invention. The drape 10 has a flexible, sterile wall 12 with opposite sides 14, 16 extending lengthwise between opposite ends 18, 20. The wall 12 is sized to cover or substantially cover a patient P (FIGS. 3 and 4), and includes a central main body portion 17 and opposite side regions 19, wherein the main body portion 17 can be constructed from a different material than the side regions 19, if desired. Otherwise, it should be recognized the central main body portion 17 and the side regions 19 could be constructed as a single, monolithic piece of the same material. By way of example and without limitation, the main body portion 17 can be constructed from a spun bond nonwoven material, while the sides regions 19 can be constructed from a thin sheet polymeric material, wherein the separate materials can be fixed to one another via any suitable bond mechanism, including a heat bonded or adhesive bonded joint, for example. The wall 12 has a cranial region 22 adapted to be releasably attached to the top region of the patient's head. Further, the wall 12 has at least one opening, and shown as a pair of opening 24, 26 located on opposite sides of a central axis 27 that bisects the cranial region 22, wherein the central axis 27 is generally parallel to the opposite sides 14, 16, and the openings 24, 26 are proximate the cranial region 22. Accordingly, the openings 24, 26 are located on opposite sides of the cranial region 22. Otherwise, as shown in FIG. 1A, wherein a drape 10′ is shown constructed in accordance with another aspect of the invention, with the same reference numerals used as above, offset with a prime symbol to identify like features, the wall 12′ can have a single, generally centrally located opening 24′ formed along the central axis 27′ adjacent the cranial region 22′ between the cranial region 22′ and the end 20′. A flexible, tubular sterile sleeve 28 (FIG. 1), 28′ (FIG. 1A) is attached to the wall 12, 12′ about each opening 24, 26, 24′, with an open end 30, 30′ (FIG. 2) of each sleeve 28, 28′ being attached in sealed relation about the entire periphery of the respective opening 24, 26, 24′, such as by being adhered to an underside of the wall 12, 12′ via any suitable adhesive or bonded joint, by way of example and without limitation, thereby forming a fluid/air-tight hermetic seal between the openings 24, 24′, 26′ and the sleeves 28, 28′. The sleeves 28, 28′ are preferably initially folded, such as in telescopic fashion, and are extendible away from the wall 12, 12′ to an optically transparent or clear free end 31.

Discussion going forward is directed to the drape 10 of FIG. 1, unless otherwise stated, though it is to be understood that the same features are present in the drape 10′. The drape 10 is used in brain surgery procedures, such as craniotomy procedures, wherein the sleeve 28 of the drape 10 needs to accommodate, by receiving therein, at least a portion of an imaging system, and in particular, as shown in FIG. 3, a stabilizer arm 32 and navigator tracker 34. With the patient P positioned on a patient support surface of the operating table, the stabilizer arm 32 is operably and adjustably attached to a head clamp 36 that has been previously fixed to the patient's head. In addition, the navigator tracker 34 is operably fixed to an end of the stabilizer arm 32. In use, with the navigator tracker attached to the stabilizer arm 32, the stabilizer arm 32 is adjusted in 3-D space about 3-axes to move the navigator tracker 34 into a precise position to image the precise location of the patient's brain. Once the navigator tracker 34 is located in the precise position, the stabilizer arm 32 is fixed against movement, thereby fixing the location of the navigator tracker 34 in its desired position. Accordingly, the navigator tracker 34 is fixed relative to the patient's head in a precise location that must be maintained throughout the surgical procedure in order for the surgeon to reliably see the precise location of the surgical instruments within the patient's cranium during the procedure.

With the patient P on the operating table and head fixed in the clamp 36, and with the navigator tracker 34 fixed in its desired position, the one-piece, impervious and imperforate drape 10 (meaning that the drape, with the sleeves 28 sealingly attached thereto is imperforate) can be disposed over the patient P to provide an upwardly facing sterile surface 38 over the entirety of the patient P and over the entire assembly of the stabilizer arm 32; navigator tracker 34, and clamp 36. As such, the region above an upper patient support surface 40 of the operating table is kept sterile, thereby facilitating the avoidance of causing infection to the patient P during the surgery.

While disposing the drape 10 over the patient P, the cranial region 22, which is fabricated of a transparent thin film, is attached to an upper surface or crown of the patient's head, such as via a self-adhesive surface on the underside of the cranial region 22. A release paper can be applied to the self-adhesive surface and then selectively removed therefrom to expose the self-adhesive surface for attachment to the patient's head. Then, the drape 10 can be unfolded along the patient's body and along the full length of the operating table to cover the entirety of the patient P and operating table, wherein the opposite ends 18, 20 of the drape 10 preferably hang over the opposite ends of the operating table and the opposite side regions 19 preferably hang over the opposite sides of the operating table. As the drape 10 is being unfolded, the respective opening(s) 24, 26, 24′ and associated sleeve 28, 28′ is registered in alignment with the navigator tracker 34 and the stabilizer arm 32. Depending on which side of the patient's head the stabilizer arm 32 is located will determine which of the openings 24, 26 is registered with the navigator tracker 34, and accordingly, the drape 10 is able to be used in procedures requiring the stabilizer arm 32 to fixed on either side of the patient's head. The sleeve 28 is preferably initially folded in telescopic fashion prior to use, and thus, upon registering the sleeve 28 with the navigator tracker 34 and the stabilizer arm 32, the drape 10 can be pressed downwardly over and about the navigator tracker 34 and the stabilizer arm 32, thereby causing the sleeve 28 to automatically unfold in telescopic fashion about the navigator tracker 34 and the stabilizer arm 32.

Upon unfolding the sleeve 28, as shown in FIG. 4, the optically clear free end 31 is brought into a snug fit about the navigator tracker 34, and thus, the navigator tracker functions as intended through the optically clear free end 31 without having to puncture the sleeve 28. It should be recognized that the entire sleeve 28 is preferably provided as a monolithic piece of tubular, optically transparent material, such as polyethylene, by way of example and without limitation, though it is contemplated that the lower body of the sleeve 28 could be formed from other than optically transparent or clear material, with only the free end 31 being formed from the optically transparent material to allow the navigator tracker 34 to function as intended. Upon unfolding the sleeve 28 about the stabilizer arm 32 and navigator tracker 34, sterility is maintained and assured above the upper surface 40 of the operating table. To assist in circumferentially gathering any excess, loose or otherwise baggy material of the sleeve 28, thereby keeping the material of the sleeve 28 from interfering with the surgical team or becoming inadvertently snagged, one or more elongate gathering members 42 can be wrapped about the sleeve 28 and fastened, such as via hook and loop (Velcro) straps, tie wraps, strings, or a self-adhesive 41 that is selectively uncovered via removal of a release paper, by way of example and without limitation. The gathering members 42 are spaced from one another axially along the length of the sleeve 28 from the base of the sleeve 28 adjacent the opening 24, 26, 24′ to the closed free end 31, as desired, to provide a suitable number of locations at which to gather the excess material of the sleeve 28. As best shown in FIG. 2, the gathering members 42 extend between opposite first and second ends 45, 47, wherein the first end 45 is fixed to the sleeve 28 and the second end 47 is a free end that can be releasably attached to the sleeve via tear tabs 43, wherein the tear tabs 43 can be perforated to facilitate tearing the tabs 43, as desired, to allow the gathering member 42 to be extended from the sleeve 28 and freely wrapped circumferentially about the stabilizer arm 32. Upon wrapping the elongate body of the gathering member 42 about the stabilizer arm 32 and causing the loose, baggy material of the sleeve 28 to be circumferentially gathered, the self-adhesive 41 can be exposed and adhered to the wall of the sleeve 28 or to the elongate body of the gathering member 42 itself to constrict the sleeve 28 in a conforming fit about the stabilizer arm 32. As such, the presence of the sleeve 28 about the stabilizer arm 32 does not interfere with the surgical team during the surgical procedure.

In addition to the sleeve 28, the drape 10 can be provided with a fluid capture extension 44 adjacent the cranial region 22. The fluid capture extension 44 is funnel-shaped and extends from an enlarged open end 46 to a reduced size closed end 48. To facilitate removal of the fluid drained into the fluid capture extension 44, a valve fitting 50 can be provided near the base of the closed end 48 for attachment to a suction hose. To facilitate keeping the open end 46 in a desired open hoop shape, a formable member 52, such as a wire, tape or foil, for example, can be laid into or other fixed adjacent the open end 46. As such, the open end 46 can be conformed, as desired, to maintain the desired shape. Further, the fluid capture extension 44 can be provided with an upper catch surface 53, such as a porous cloth, wherein the catch surface 53 extends across the full opening adjacent the open end 46 to catch non-fluid matter, e.g. surgical instruments, from falling to the closed end 48 of the fluid capture extension 44. As such, if the surgeon accidentally drops an instrument, the instrument will be readily caught by the catch surface 53, whereupon the instrument can be readily retrieved.

In addition, the drape 10 can be provided with a plurality of fasteners 54, such as self-adhesive strips or hook and loop fasters (Velcro), adjacent the cranial region 22. The fasteners 54 can include a strap 56 with one of the hook or loop, while the upper surface of the drape 10 can have a fastener pad 57 including the other of the hook or loop. In use, the strap 56 can be detached from the upper surface of the drape 10, and a tube or line can be run between the strap 56 and the upper surface of the drape, whereupon the strap 56 can be pressed over the tube or line and releasably fastened to the upper surface 57 of the drape 10. As such, a plurality of tubes or lines can be organized and releasably fixed in place during the surgical procedure to keep the tubes or lines from interfering with the procedure. Accordingly, the drape 10 provides a plurality of functional elements that facilitate maintaining sterility in the surgical arena, while also facilitating the organization of various lines used in the surgical procedure. Further yet, as shown in FIG. 4, the drape 10 is provided with one or more pockets 58 that can be selectively adhered to the outer surface of the drape 10, such as near the cranial end 18, wherein the pockets 58 provide ready storage pouches for surgical instruments and the like. The pockets 58 can be readily affixed to the outer surface via one or more self-adhesive strips 60 upon removing a release paper therefrom. As such, further functionality can be provided by the drape 10.

Many modifications and variations of the present invention are possible in light of the above teachings. It is, therefore, to be understood that the invention may be practiced otherwise than as specifically described, and that the scope of the invention is defined by any ultimately allowed claims.

What is claimed is:

1. A sterile craniotomy drape, comprising:
a flexible, sterile wall with opposite edges extending lengthwise between opposite ends, said wall being sized to substantially cover a patient;
said wall having a cranial region adapted to be attached to the patients head and at least one opening proximate said cranial region; and
a flexible, tubular sterile sleeve having an open end attached to said wall about said at least one opening, said sleeve being extendible away from said wall to a free end.

2. The sterile craniotomy drape of claim 1 wherein said wall has a pair of said at least one openings, each of said openings being on opposite sides of said cranial portion.

3. The sterile craniotomy drape of claim 1 wherein said free end is closed.

4. The sterile craniotomy drape of claim 1 wherein said sleeve unfolds telescopically.

5. The sterile craniotomy drape of claim 1 wherein said sleeve is optically transparent.

6. The sterile craniotomy drape of claim 1 further including at least one gathering member on said sleeve, said at least one gathering member being adapted to maintain material of said sleeve in a circumferentially gathered configuration.

7. The sterile craniotomy drape of claim 6 wherein said sleeve has a plurality of said gathering members.

8. The sterile craniotomy drape of claim 6 wherein said at least one gathering member extends between opposite first and second ends, said first end being fixed to said sleeve and said second end having a fastener configured for releasable attachment to at least one of said sleeve and said gathering member.

9. The sterile craniotomy drape of claim 8 wherein said fastener is a self-adhesive.

10. The sterile craniotomy drape of claim 8 further including a tear tab releasably attaching said second end of at least one gathering member to said sleeve.

11. A method of simultaneously draping a sterile barrier over a patient and a navigation tracker for a craniotomy procedure, comprising:
providing a sterile patient drape having an opening with a sterile tubular sleeve attached about the opening;
registering the tubular sleeve with the navigation tracker and covering the patient with the patient drape; and
unfolding the tubular sleeve about the navigator tracker.

12. The method of claim 11 further including gathering material of the tubular sleeve about the stabilizer arm and constraining the material in its gathered configuration with a gathering member.

13. The method of claim 12 further including removing a release paper from a self-adhesive on the gathering member and releasably fixing the self-adhesive to one of the sleeve and gathering member.

14. The method of claim 12 further including extending a free end of the gathering member from the sleeve and wrapping the gathering member circumferentially about the sleeve.

15. The method of claim 14 further including tearing a tear tab to allow the free end of the gathering member to be extended from the sleeve.
